(19) United States
(12) Patent Application Publication
(10) Pub. No.: US 2021/0228891 A1
(43) Pub. Date: Jul. 29, 2021

(54) Method for Implanting an Implantable Medical Device

(71) Applicant: BIOTRONIK SE & Co. KG, Berlin (DE)

(72) Inventors: Thomas Doerr, Berlin (DE); Gernot Kolberg, Berlin (DE)

(21) Appl. No.: 17/155,271

(22) Filed: Jan. 22, 2021

Related U.S. Application Data
(60) Provisional application No. 62/964,663, filed on Jan. 23, 2020.

(51) Int. Cl. A61N 1/375; A61N 1/37

(52) U.S. Cl. CPC A61N 1/37512; A61N 1/3702; A61N 1/3706; A61N 1/3756

(57) ABSTRACT

A method for implanting an implantable medical device into a body of a human patient in need of such implantation is disclosed. This method comprises the following steps: a) performing a single skin incision into the patient's skin in the abdominal region of the patient; b) moving the implantable medical device through the skin incision under the patient's skin into the patient's abdomen; c) moving a first electrode and a second electrode through the skin incision under the patient's skin into the thoracic region of the patient; d) connecting the first electrode and the second electrode to the implantable medical device if the first electrode and the second electrode are not yet connected to the implantable medical device; and e) closing the skin incision.

### CROSS-REFERENCE TO RELATED APPLICATIONS

This patent application claims the benefit of and priority to co-pending U.S. Provisional Patent Application No. 62/964/663, filed on Jan. 23, 2020 which is hereby incorporated by reference in its entirety

### TECHNICAL FIELD

The present invention relates in an aspect to a method for implanting an implantable medical device into a body of a human patient in need of such implantation. A further aspect of the present invention relates to a kit for implanting an implantable medical device into a body of a human patient. A further aspect of the present invention relates to a method for treating a cardiac arrhythmia of a human patient with a cardiac pacing arrangement.

### BACKGROUND

Currently available subcutaneous implantable cardioverter-defibrillator (S-ICD) systems consist of a subcutaneously implanted three-polar electrode, which is preferably parasternally implanted on the left side of the body of the patient, and a generator, which acts as a counter electrode for sending and defibrillation and is submuscularly implanted laterally in the area of the left thorax of the patient. Disadvantages of this solution are a comparatively complicated submuscular implantation procedure with the necessity of general anesthesia or deep sedation, which requires the presence of an anesthetist and is associated with corresponding complications (e.g. anesthesia complications, pain treatment after implantation, increased risk of infection due to the comparatively large traumatization in the submuscular area).

Implantation of such S-ICD system requires two or three skin incisions, These incisions are an additional risk of infection for the patient. There are also cosmetic disadvantages of the existing systems since at least two scars will result from implantation. One scar will be present at a pocket of the generator and another scar at the lower end of the sternum. Typically, a third scar will result at a fixation of the distal end of the electrode at the upper end of the sternum in the cleavage area.

The present invention is directed toward overcoming one or more of the above-mentioned problems, though not necessarily limited to embodiments that do.

### SUMMARY OF THE INVENTION

It is an object of the present invention to provide an implantation method for an implantable medical device, the implantation method overcoming at least some of the disadvantages of prior art implantation methods. A further object of the present invention is to provide a kit being appropriate for carrying out such an implantation method. A further object of the present invention is to provide a method of treatment of a cardiac arrhythmia of a patient being less complicated than treatment methods known from prior art.

At least these objects are addressed, in an aspect, by a method for implanting an implantable medical device into a body of a human patient in need of such implantation. The human patient has a heart, a skin, a thorax and an abdomen. The thorax is located in a thoracic region of the patient. Likewise, the abdomen is located in an abdominal region of the patient.

The implantable medical device is configured to sense electrical signals from the patient's heart and optionally to stimulate the patient's heart with electrical signals. Thus, the implantable medical device can be, in a first aspect, a diagnostic device. In a second aspect, the implantable medical device is not only a diagnostic device, but also a therapeutic device.

The method addressing at least one of the objects underlying the present invention comprises the steps explained in the following.

First, a single skin incision is made into the patient's skin in the abdominal region of the patient. Typically, this skin incision is made in a lower area of the abdominal region of the patient or below the thorax of the patient By this skin incision, an interior of the abdominal region of the patient becomes accessible for a surgeon.

Then, the implantable medical device is moved through the skin incision under the patients skin into the patient's abdomen. Furthermore, a first electrode and a second electrode are moved through the skin incision under the patient's skin into the thoracic region of the patient.

The first electrode and/or the second electrode might already be connected to the implantable medical device upon implanting the electrode(s). However, it is likewise possible that a connection between the first electrode and/or the second electrode on the one hand and the implantable medical device on the other hand needs still to be established. Therefore, the first electrode and the second electrode are connected to the implantable medical device if the first electrode and the second electrode have not yet been connected to the implantable medical device beforehand. It is possible that one of the first electrode and the second electrode is pre-assembled with the implantable medical device and thus has already been connected with the implantable medical device. In such a case, it is only necessary to connect the other of the first and second electrode with the implantable medical device. It is, in an embodiment, also possible that none of the first and second electrodes are pre-assembled with the implantable medical device and thus need both to be connected with the implantable medical device after having been moved through the skin incision into the patient's abdomen.

Finally, the skin incision is closed. This can be made by a surgical suture, by cramping, by gluing or by any other technique appropriate for closing skin incisions well known to the person skilled in the art.

This implantation method makes it possible to implant a medical device through a single skin incision. No further incisions are necessary. Therefore, only a single scar will result so that both the risk of infection and cosmetic disadvantages are significantly reduced with respect to prior art implantation techniques.

The implantable medical device is typically located in a central abdominal area so that the patient has a much higher wearing comfort compared to implantable medical devices having been implanted according to prior art techniques. This is due to the fact that according to prior art techniques, subcutaneously implantable medical devices are typically implanted below the arm of the patient. There, it disturbs the patient upon any movements of the arm. The presently claimed implantation method, however, enables an implantation on the muscles in the abdominal region where the implantable medical device disturbs the patient much less than under the arm.

In an embodiment, the method further comprises a step of preparing a pocket in the abdominal region of the patient. In this context, the pocket is configured to receive the implantable medical device after the implantable medical device has been moved into the patient's abdomen. Thus, the pocket serves as a receptacle for the implantable medical device. The pocket helps in avoiding an undesired dislocation of the implantable medical device once being in its implanted state, in particular in a subcutaneous abdominal region of the patient.

The skin incision serves for subcutaneously implanting the implantable medical device and for subcutaneously implanting the first and second electrodes connected or to be connected to the implantable medical device. However, the concrete location of the implantable medical device can be adapted to the patients need or in reaction of specific anatomical circumstances within certain framework conditions.

In an embodiment the implantable medical device is implanted within fatty tissue of the patient.

In another embodiment, the implantable medical device is implanted between fatty tissue and a muscular fascia of the patient.

In another embodiment, the implantable medical device is implanted onto an abdominal muscle of the patient below or within a muscular fascia of the abdominal muscle.

In another embodiment, the implantable medical device implanted into an abdominal muscle of the patient.

In another embodiment, the implantable medical device is implanted left to the xiphoid process below the patient's thorax. At this implantation site, the implantable medical device will disturb the patient only to a particularly small extent.

All of the precedingly explained implantation sites—in particular the non-intramuscular implantation sites—require a much less severe intervention than the implantation techniques known from prior art. Therefore, it is well conceivable that such an implantation can be carried out without total anesthesia, but only under local anesthesia. Therefore, the side-effects of such an implantation will be much less severe than in case of implantation methods known from prior art.

In an embodiment, the first electrode and the second electrode are placed below the patient's skin on the patient's thorax. In such a case, the first electrode and the second electrode are located at a first distance from each other. The first distance amounts to at least 5 cm, in particular at least 7 cm, in particular at least 10 cm, in particular at least 12 cm, in particular at least 15 cm, in particular at least 20 cm, in particular at least 2.5 cm, in particular at least 30 cm, in particular at least 40 cm. A first distance in a range between 5 cm and 40 cm or in any other interval of the precedingly mentioned lower thresholds of the first distance is particularly appropriate for placing the first electrode and the second electrode on the patient's thorax.

In an embodiment, the first electrode and the second electrode are placed below the patient's skin on the patient's thorax such that a center of an electrode pole of the first electrode is located on the right thoracic side of the patient between the third rib and the seventh rib of the patient and that a center of an electrode pole of the second electrode is located on the left thoracic side of the patient between the forth rib and the seventh rib of the patient.

In an embodiment, the first electrode and the second electrode are placed below the patient's skin on the patient's thorax in a V-like arrangement. In such an arrangement, the longitudinally extending electrodes are arranged to each other in an angular manner, wherein an angle between a longitudinal direction of extension of the first electrode and a longitudinal direction of extension of the second electrode lies in a range of between 20° and 170°, in particular between 30° and 160°, in particular between 40° and 150°, in particular between 50° and 140°, in particular between 60° and 130°, in particular between 70° and 120°, in particular between 80° and 110°, in particular between 90° and 100°.

As outlined above, it is possible to connect the first electrode and/or the second electrode to the implantable medical device prior to inserting the medical device through the skin incision into the abdominal region of the patient or after having inserted the implantable medical device through the skin incision into the abdominal region of the patient. In an embodiment, it is also possible that at least one of the first electrode and the second electrode is fixedly connected to the implantable medical device. Then, no additional connecting step is necessary to establish an electric connection between the first electrode and or the second electrode on the one hand and the implantable medical device on the other hand.

In an embodiment, more than two electrodes are connected to the implantable medical device. In this embodiment, at least one further electrode is moved through the skin incision under the patient's skin into the thoracic region of the patient. In this case, the at least one further electrode is connected or is intended to be connected to the implantable medical device after having been implanted itself. Thus, an aspect of the presently claimed implantation method relates to an implantation of the medical device and 3, 4, 5 or even more electrodes so as to be able to address basically all medical conditions to be treated with the implantable medical device.

In an embodiment, the implantation method is carried out such that the implantation results in at least one of the anatomical implantation conditions explained in the following. In a first variant, the implantable medical device is placed left under the xiphoid process between subcutaneous fat and abdominal musculature or below abdominal musculature. Then, the implantable medical device is on the one hand well protected against the environment of the patient, but still does not severely interact with the musculature of the patient.

In a second variant, the first electrode is placed subcutaneously along the sternum, either mid sternal, left parasternal or right parasternal.

In a third variant, the second electrode is placed onto the left side of the patient's thorax (but still under the patient's skin) and terminates at the area of the mid to dorsal part of latissimus dorsi muscle and/or the serratus anterior muscle and is placed either between this two muscles or between the rib cage and the serratus anterior muscle.

In an embodiment, the first variant and the second variant are combined. In a further embodiment, the first variant and the third variant are combined. In a further embodiment, the second variant and the third variant are combined. In a further embodiment, the first variant, the second variant and the third variant are combined.

In an embodiment, the first electrode and/or the second electrode are moved into the thoracic region of the patient by a tunneling process. In this context, the tunneling process comprises the steps explained in the following.

First, a tunneling tool located within a tube provided. The tube series as insertion catheter.

Then, the tunneling tool and the tube are moved together through the skin incision towards the intended implantation site of the first electrode or the second electrode until a distal end of the tube is located at the intended implantation site.

Then, the tube is kept in place. However, the tunneling tool is removed from the interior of the tube. Thus, the tunneling is moved proximally out of the tube.

Afterwards, the first electrode or the second electrode is moved through the tube (instead of the tunneling tool) towards the intended implantation site of the respective electrode. This movement takes place until the respective electrode exits the tube at the intended implantation site. Optionally, the electrode can then be fixed at its implantation site.

In any case, the first electrode or the second electrode (depending on which electrode has been moved through the tube) is kept in place, wherein the tube is now removed from the patient's body (e.g., by a movement of the tube in a proximal direction). Upon removal of the tube, the electrode remains in the previously prepared path within the body of the human patient.

Finally, the tube is removed from the electrode. If the electrode is not yet connected to the implantable medical device, this removing can take place by pulling the tube over a plug connector at the proximal end of the electrode. In such a case, the diameter of the tube needs to be sufficiently big to be pulled over the plug connector of the electrode.

In another embodiment, the tube is removed from the electrode by peeling the tube from the electrode. Such peeling can be achieved, e.g., by configuring the tube such that it comprises at least one portion with reduced wall thickness. This portions defines a predetermined breaking point of the tube at which the tube can be split up into two or more parts by a process that is known to the person skilled in the art as peeling.

In another embodiment, the tube is slit with a slit tool and removed from the electrode in its slit state.

Appropriate materials for the tube are different plastics. In an embodiment, the tube comprises at least partially a mesh tubing.

The precedingly described tunneling process can also be denoted as a pushing technique since the electrode is pushed through the tube once the tube has been correctly positioned within the patient's body towards the intended implantation site of the electrode. Thereby, the electrode is pushed along an implantation path that has been formed by the tunneling tool.

In another embodiment, the first electrode and/or the second electrode is/are moved into the thoracic region of the patient by a different tunneling process. In this context, the tunneling process comprises the steps explained in the following.

First, a tunneling tool and a tube are provided. In this context, the tunneling tool is configured to fit into an interior of the tube. The tunneling tool further comprises an electrode receiving portion in a distal region of the tunneling tool, in particular in a distal end region of the tunneling tool. This electrode receiving portion can be realized, e.g., by a hook being open to the distal side of the tunneling tool.

Then, the first electrode or the second electrode is connected to the tunneling tool at the electrode receiving portion. For this purpose, the first electrode or the second electrode might comprise an engaging element (like a ring or a loop) that can be engaged by the electrode receiving portion of the electrode.

Once the electrode is connected to the tunneling tool, both the tunneling tool and the electrode can be inserted into an interior of the tube. Then, the tube, the tunneling tool and the respective electrode can be moved together through the skin incision towards the intended implantation site of the respective electrode until a distal end of the tube is located at the intended implantation site.

At this point, the respective electrode that has been connected to the tunneling tool beforehand is now disconnected from the tunneling tool. Thus, it can be moved inside the tube independently on the tunneling tool.

The respective electrode that has been guided through the tube to its intended implantation site is kept in place, wherein the tunneling tool and the tube are removed from the patient's body.

Finally, the tube is removed from the electrode. In this context, the different removing techniques explained above with respect to the alternative tunneling process can also be applied in this case.

This tunneling process can also be denoted as a towing technique.

In an aspect, the present invention relates to a kit for implanting, an implantable medical device into a body of a human patient in need of such implantation. This kit is particularly appropriate to be used in connection with the precedingly described implantation method.

The kit comprises an implantable medical device configured to sense electrical signals from the heart of the patient and optionally to stimulate the patient's heart with electrical signals. In this context, the implantable medical device has at least two electrode connectors. Each of the electrode connectors is configured and intended to receive a connector plug of an electrode so that two electrodes can be connected to the implantable medical device.

The kit further comprises a first electrode being already connected to the implantable medical device or being connectable to the first of the electrode connectors of the implantable medical device. The kit further comprises a second electrode being already connected to the implantable medical device or being connectable to a second of the electrode connectors of the implantable medical device.

The kit further comprises at least one tunneling tool configured to prepare an implantation path for at least one of the first electrode and the second electrode. Typically, the tunneling tool is used to prepare a first implantation path for the first electrode and a second implantation path for the second electrode. However, it is also possible to include two different tunneling tools into the described and claimed kit.

The implantable medical device is a device intended to be permanently implanted into the body of a human patient. It comprises diagnostic and/or therapeutic functions. The implantable medical device comprises a housing that can serve as electrode. Alternatively, the housing can comprise one or more electrode surfaces. Alternatively, the housing can be completely electrically insulated. A power source is integrated into the implantable medical device, in particular in the housing of the implantable medical device. In an embodiment, the power supply is a rechargeable power supply, such as a rechargeable battery. The implantable medical device comprises one or more input amplifiers with the help of which physiologic signals sensed by the electrodes can be processed.

In an embodiment, the implantable medical device is a subcutaneously implantable cardioverter-defibrillator (S-ICD) or a device for cardiac resynchronization therapy (CRT-D). In an embodiment, the housing of the implantable medical device remains mostly passive during a defibrillation shock delivered by at least one of the electrodes of the implantable medical device. “Passive” in this context means that the housing does not participate or does participate only to a very small extent in such defibrillation shock delivered by the implantable medical device. Typically, the housing is configured such to be available for sensing electrical signals of the patient's body, in particular of the patient's heart.

The first electrode and or the second electrode are longitudinally extending permanent implants comprising at the proximal end a possibility for connecting the electrodes to the implantable medical device. Typically, a connector plug is provided at the proximal end of the respective electrode.

One or more electrode surfaces or electric poles and/or sensors and or actuators by means of which physiological signals can be sensed from specific body regions of the patient and/or site-specific therapies can be delivered to the patient, are located at the distal end of the electrode and or along the electrode. In an embodiment, the first electrode and or the second electrode comprises at least one electrode pole in the form of a coil. Such a coil is particularly appropriate to deliver an electric shock to the patient for defibrillation purposes.

In an embodiment, at least one shock electrode surface (i.e., an electrode pole configured to deliver a defibrillation shock) is present on the first electrode and/or the second electrode. In an embodiment, at least one sensing electrode pole for sensing the potential of the heart muscle and optionally for measuring an impedance is provided on the first electrode and/or the second electrode. In an embodiment, the first electrode and/or the second electrode comprise both a shock electrode surface and a sensing electrode pole. In an embodiment, the first electrode and the second electrode are identical in construction.

In an embodiment, the therapeutic action of at least one of the first and second electrode is chosen from the group consisting of a current pulse for stimulating the patient's heart, a current shock for defibrillating the patient's heart, and direct current (DC) or alternating current (AC) for conditioning the cardiac tissue or for delivering a medicament.

In an embodiment, the tunneling tool is a longitudinally extending rod. In an embodiment, the tunneling tool comprises or entirely consists of metal (e.g., stainless steel), plastic (e.g., polyether ether ketone (PEEK) or polycarbonate (PC)) or a carbon fiber material. In an embodiment, the tunneling tool is deformable by ductile deformation. Then, the tunneling tool can be bent such to adopt one or more curvatures with the same or different radii. Thus, it is possible to shape the tunneling tool such to be adapted for a path which is to be followed by the tunneling tool under the patient's skin that describes patient-individual radii (e.g., the shape of the thorax of the patient).

In an embodiment, the tunneling tool is delivered in a pre-shaped manner.

In an embodiment, the tunneling tool has at or near its proximal end a grip-like structure facilitating gripping, moving forwards (in a distal direction), moving backwards (in a proximal direction) or rotating the tunneling tool around its longitudinal axis. In an embodiment, the grip-like structure enables as user to recognize the concrete orientation of the tunneling tool from the position of the grip-like structure. Thus, the grip-like structure is, in an embodiment, not realized in form of a rotationally symmetric grip, but in form of a rotationally asymmetric grip.

In an embodiment, the distal end of the tunneling tool is a blunt end (butt) to enable an atraumatic advancement of the tunneling tool. In an embodiment, the distal end of the tunneling tool has an approximately hemispherical shape.

Generally, it is possible to directly move the tunneling tool together with the first electrode or the second electrode under the skin of the patient. An even more comfortable implantation can be achieved with the help of an insertion catheter. Therefore, the kit comprises, in an embodiment, an insertion catheter configured to receive the tunneling tool in its interior during advancement of the tunneling tool through the body of the human patient. In this context, the insertion catheter serves for preparing an implantation path for at least one of the first electrode and the second electrode. Then, the respective electrode can be inserted into the human body along the prepared implantation path. Such a procedure enables a particularly low-resistance advancement of the respective electrode and reduces the risk of damaging the electrodes during their advancement under the skin of the patient.

An aspect of the present invention relates to method for treating a cardiac arrhythmia of a human patient with a cardiac pacing arrangement. In this context, the cardiac pacing arrangement comprises an implantable medical device. This implantable medical device is configured to sense electrical signals from the patient's heart and to stimulate the patient's heart with electric signals. Furthermore, the implantable medical device has at least two electrode connectors that serve for establishing an electrical contact to at least two electrodes.

The cardiac pacing arrangement further comprises a first electrode connected to the first of the electrode connectors of the implantable medical device and a second electrode connected to the second of the electrode connectors of the in medical device. This connection is typically established with the help of a connector plug of the respective electrode.

What is special about the claimed method of treatment is that the cardiac pacing arrangement has been implanted into the human patient by a single skin incision into the patient's skin in an abdominal region of the patient. Thus, this method of treatment relies on a subcutaneous implantation of the implantable medical device through a single incision. In doing so the according implantation is much less invasive for the patient than prior art implantation techniques. Consequently, the overall method of treatment is connected to significantly less side-effects than methods of treatment known from prior art.

In an embodiment, the first electrode and the second electrode are implanted under the patient's skin on the thorax of the patient. In an embodiment, the first electrode and the second electrode are implanted under the patient's skin between muscles of the patient in the thoracic region. In an embodiment, the first electrode and the second electrode are implanted under the patient's skin under muscles of the patient in the thoracic region. In each of the precedingly explained embodiments, a vector of an electric field between the first electrode and the second electrode crosses the biggest possible muscle mass of the patient's heart. Then, it is particularly easy to stimulate the patient's heart with electric pukes delivered by either of the first electrode and the second electrode and by using the other of the first electrode and the second electrode as counter electrode.

In an embodiment, the implantable medical device carries out the steps explained in the following for delivering a cardiac therapy to the patient's heart.

In a first step, electrical signals of the patient's heart are sensed with at least one of the first electrode, the second electrode and a housing of the implantable medical device. These electrical signals of the patient's heart can be depicted, e.g., in form of an electrocardiogram.

The sensed electrical signals are evaluated to detect a cardiac rhythm of the patient's heart requiring therapy. Typically, such an evaluation takes place continuously during sensing of the electrical signals of the patient's heart.

If the implantable medical device has detected a cardiac rhythm that requires therapy, at least one of the first electrode, the second electrode and the housing of the implantable medical device is used to deliver an electrical pulse to the patient's heart. This electrical pulse can be delivered once or—in form of a plurality of electrical pulses—in a cyclic manner. In this context, the electrical pulse is appropriate to therapeutically act upon the patient's heart.

In an embodiment, an anti-bradyarrhythmia therapy is applied by the implantable medical device. In such a case, a plurality of low-energy pulses is delivered. Such therapy is typically applied if a bradyarrhythmia has been detected from the sensed electrical pulses of the patient's heart. If, however, a tachyarrhythmia has been detected, an anti-tachyarrhythmia therapy is applied by the implantable medical device. This is done by delivering a single high energy pulse to the patient's heart. Such a single high-energy pulse typically serves for defibrillating the patient's heart so as to enable the patient's heart to return hack to a normal cardiac rhythm.

All embodiments, variants and aspects of the described methods can be combined in any desired way and can be transferred in any combination to the respective other method and to the described kit. Furthermore, all variants, aspects and embodiments of the described kit can be combined in any desired way and can be transferred in any combination to the described methods.

Additional features, aspects, objects, advantages, and possible applications of the present invention will become apparent from a study of the exemplary embodiments and examples described below, in combination with the Figure and the appended claims.

### BRIEF DESCRIPTION OF THE DRAWINGS

Further details of aspects of the present invention will be described with respect to an exemplary embodiment and an accompanying Figure. In this context,

FIG. 1 schematically shows an exemplary embodiment of a cardioverter-defibrillator subcutaneously implanted by a single skin incision;

FIG. 2 shows a kit for implanting an implantable medical device into a body of a human patient in need of such implantation; and

FIG. 3 schematically shows the abdomen region illustrating the incisions, paths and pocket(s) utilized for implanting an implantable medical device into a body of a human patient in need of such implantation.

### DETAILED DESCRIPTION

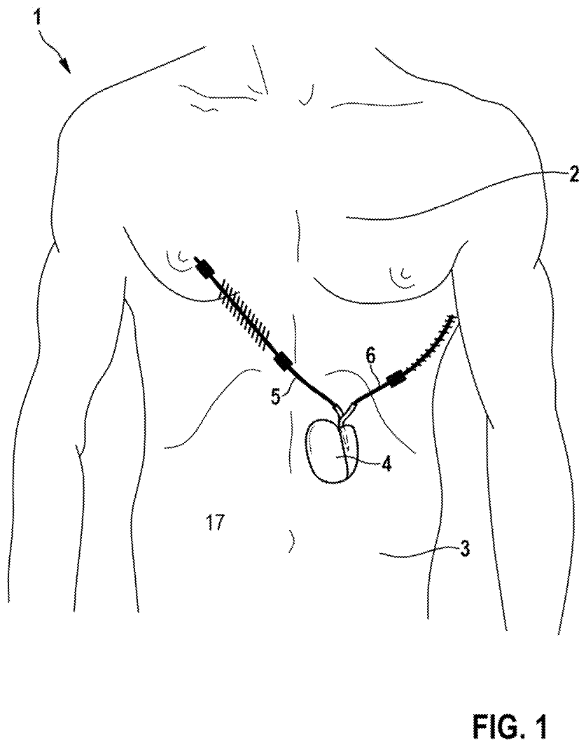

FIG. 1 knows as schematic depiction of a patient 1 having a thorax 2 and an abdomen 3. The skin 17 of the patient 1 is partially cut in FIG. 1 to allow a view onto an implantable cardioverter-defibrillator 4 that is implanted below the skin 17 of the patient in the region of the abdomen 3. The cardioverter-defibrillator 4 serves as implantable medical device, it comprises two electrode connectors, wherein a first electrode 5 is connected to a first of these electrode connectors and a second electrode 6 is connected to a second of these electrode connectors.

The first electrode 5 extends below the skin 1 of the patient 1 towards the right side of the thorax 2, whereas the second electrode 6 extends from the cardioverter-defibrillator 4 towards the left side of the thorax 2 of the patient 1. The first electrode 5 and the second electrode 6 are arranged in a V-like manner, wherein an angle of approximately 90° is established between a longitudinal extension direction of the first electrode 5 and a longitudinal extension direction of the second electrode 6.

The cardioverter-defibrillator 4, the electrode 5 and the second electrode 6 have been subcutaneously implanted to the patient 1 by using a single skin incision through which the cardioverter-defibrillator 4, the first electrode 5 and the second electrode 6 have been moved into an abdominal region 3 of the patient 1. The advancement and fixation of the first electrode 5 and the second electrode 6 at the respective intended implantation sites on the thorax 2 of the patient 1 have been accessed subcutaneously through this single skin incision. Therefore, the side-effects of the implantation of the cardioverter-delibrillator 4 with the first electrode 5 and the second electrode 6 are much smaller than in case of classic implantation techniques requiring two or even three skin incisions.

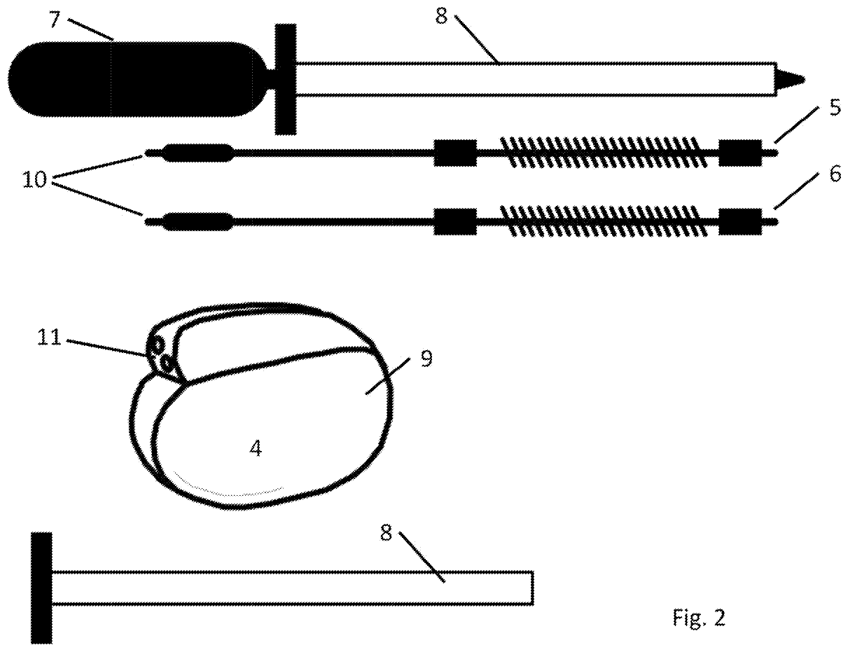

FIG. 2 illustrates a kit for implanting an implantable medical device into a body of a human patient in need of such implantation. This kit is particularly appropriate to be used in connection with the herein described implantation method.

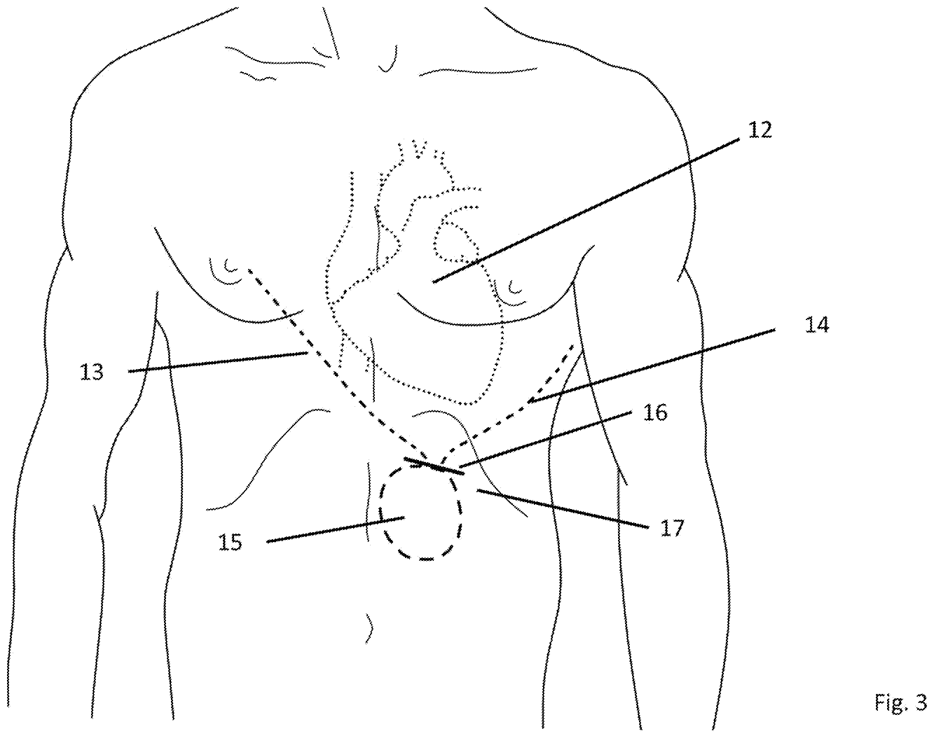

Referring to FIGS. 2-3, the kit comprises an implantable medical device 4 having a housing 9 and configured to sense electrical signals from the heart 12 of the patient and optionally to stimulate the patient's heart 12 with electrical signals. In this context, the implantable medical device 4 has at least two electrode connectors 11 (e.g., female plugs). Each of the electrode connectors 11 is configured and intended to receive a connector plug 10 of an electrode 5, 6 so that two electrodes 5, 6 can be connected to the implantable medical device 4.

The kit further comprises a first electrode 5 being already connected to the implantable medical device 4 or being connectable to the first of the electrode connectors 11 of the implantable medical device 4. The kit further comprises a second electrode 6 being already connected to the implantable medical device 4 or being connectable to a second of the electrode connectors 11 of the implantable medical device 4.

The kit further comprises at least one tunneling tool 7 configured to prepare an implantation path for at least one of the first electrode 5 and the second electrode 6. Typically, the tunneling tool 7 is used to prepare a first implantation path 13 for the first electrode 5 and a second implantation path 14 for the second electrode 6. However, it is also possible to include two or more different tunneling tools into the described and claimed kit.

The kit further comprises at least two insertion catheters 8 configured to receive the tunneling tool 7 in its interior during an advancement of the tunneling tool 7 through a body of a human patient for preparing the implantation paths 13, 14 for at least one of the first electrode 5 and the second electrode 6. In a preferred embodiment, one insertion catheter 8 is required for each implanted electrode. During implantation, the tunneling tool 7, with the mounted insertion catheter 8 inserted thereon. The tunneling tool 7 is advanced along implantation path 13 for the first electrode 5. After proper placement, the tunneling tool 7 is removed and the first electrode 5 is implanted via the insertion catheter 8. After the first electrode 5 is inserted, the insertion catheter 8 is slit or split in order to be able to remove it and leave the first electrode 5 in place. A second insertion catheter 8 is inserted on the tunneling tool 7, and the tunneling tool 7 is advanced along implantation path 14 for the second electrode 6. After proper placement, the tunneling tool 7 is removed and the second electrode 6 is implanted via the insertion catheter 8. After the second electrode 6 is inserted, the insertion catheter 8 is slit or split in order to be able to remove it and leave the second electrode 6 in place. This process is repeated for each electrode that is implanted.

As shown in the top view of FIG. 2, the insertion catheter 8 can be premounted on the tunneling tool 7. However, the insertion catheter 8 does not have to be premounted, as shown in the bottom view of FIG. 2. But since the physician will have to mount the insertion catheter 8 to the tunneling tool 7, it makes it easier for the physician if the two are premounted.

FIG. 3 schematically shows the abdomen region illustrating the incisions, paths and pocket(s) utilized for implanting an implantable medical device into a body of a human patient in need of such implantation. Referring to FIGS. 1-3, a method for implanting an implantable medical device into a body of a human patient 1 in need of such implantation is provided, the human patient 1 having a heart 12, a skin 17, a thorax 2 and an abdomen 3, wherein the thorax 2 is located in a thoracic region of the patient 1 and the abdomen 3 is located in an abdominal region of the patient 1. The implantable medical device 4 is configured to sense electrical signals from the patient's heart and optionally to stimulate the patient's heart with electrical signals.

The method includes the following steps: a) performing a single skin incision 16 into the patient's skin 17 in the abdominal region 3 of the patient 1; b) moving the implantable medical device 4 through the skin incision 16 under the patient's skin 17 into the patient's abdomen 3; c) moving a first electrode 5 and a second electrode 6 through the skin incision 16 under the patient's skin into the thoracic region 2 of the patient 1; d) connecting the first electrode 5 and the second electrode 6 to the implantable medical device 4 if the first electrode 5 and the second electrode 6 are not yet connected to the implantable medical device 4; and e) closing the skin incision 16.

In an embodiment, the method further comprises the step of preparing a pocket 15 in the abdominal region 3 of the patient 1. In this context, the pocket 15 is configured to receive the implantable medical device 4 after the implantable medical device 4 has been moved into the patient's abdomen 3. Thus, the pocket 15 serves as a receptacle for the implantable medical device 4. The pocket 15 helps in avoiding an undesired dislocation of the implantable medical device 4 once being in its implanted state, in particular in a subcutaneous abdominal region 3 of the patient 1.

The skin incision 16 serves for subcutaneously implanting the implantable medical device 4 and for subcutaneously implanting the first 5 and second 6 electrodes connected or to be connected to the implantable medical device 4. However, the concrete location of the implantable medical device 4 can be adapted to the patients need or in reaction of specific anatomical circumstances within certain framework conditions.

The at least one tunneling tool 7 configured to prepare an implantation path for at least one of the first electrode 5 and the second electrode 6. Typically, the tunneling tool 7 is used to prepare a first implantation path 13 for the first electrode 5 and a second implantation path 14 for the second electrode 7. However, it is also possible to include two different tunneling tools to prepare the two paths 13, 14.

In a further form, the insertion catheter 8 is configured to receive the tunneling tool 7 in its interior during an advancement of the tunneling tool 7 through a body of a human patient 1 for preparing the implantation paths 13, 14 for at least one of the first electrode 5 and the second electrode 6, respectively.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof Additionally, the disclosure of a range of values is a disclosure of every numerical value within that range, including the end points.

What is claimed is:

1. A method for implanting an implantable medical device into a body of a human patient in need of such implantation,
the human patient having a heart, a skin, a thorax and an abdomen, wherein the thorax is located in a thoracic region of the patient and the abdomen is located in an abdominal region of the patient,
the implantable medical device being configured to sense electrical signals from the patient's heart and optionally to stimulate the patient's heart with electrical signals,
the method comprising the following steps:
a) performing a single skin incision into the patient's skin in the abdominal region of the patient;
b) moving the implantable medical device through the skin incision under the patient's skin into the patient's abdomen;
c) moving a first electrode and a second electrode through the skin incision under the patient's skin into the thoracic region of the patient;
d) connecting the first electrode and the second electrode to the implantable medical device if the first electrode and the second electrode are not yet connected to the implantable medical device; and
e) closing the skin incision.

2. The method according to claim 1, further comprising preparing a pocket in the abdominal region of the patient, the pocket being configured to receive the implantable medical device after the implantable medical device has been moved into the patient's abdomen.

3. The method according to claim 1, wherein the implantable medical device is implanted within fatty tissue of the patient.

4. The method according to claim 1, wherein the implantable medical device is implanted between fatty tissue of the patient and a muscular fascia of the patient.

5. The method according to claim 1, wherein the implantable medical device is implanted onto an abdominal muscle of the patient below or within a muscular fascia of the abdominal muscle.

6. The method according to claim 1, wherein the implantable medical device is implanted into an abdominal muscle of the patient.

7. The method according to claim 1, wherein the implantable medical device is implanted left to the xiphoid process below the patient's thorax.

8. The method according to claim 1, wherein the first electrode and the second electrode are placed below the patient's skin on the patient's thorax in a first distance from each other, wherein the first distance amounts to at least 5 cm.

9. The method according to claim 1, wherein the first electrode and the second electrode are placed below the patient's skin on the patient's thorax in a V-like arrangement, wherein an angle between a longitudinal direction of extension of the first electrode and a longitudinal direction of extension of the second electrode lies in a range of between 20 degrees and 170 degrees.

10. The method according to claim 1, wherein at least one of the first electrode and the second electrode is fixedly connected to the implantable medical device.

11. The method according to claim 1, wherein at least one further electrode is moved through the skin incision under the patient's skin into the thoracic region of the patient, the at least one further electrode being connected or intended to be connected to the implantable medical device.

12. The method according to claim 1, wherein the method for implanting results in at least one of the following implantation conditions:
i) the implantable medical device is placed left below the xiphoid process between subcutaneous fat and abdominal musculature or below abdominal musculature;
it) the first electrode is placed subcutaneously along the sternum, either mid sternal, left parasternal or right parasternal placed;
iii) the second electrode is placed onto the left side of the patient's thorax and terminates at the area of the mid to dorsal part of latissimus dorsi muscle and/or the serratus anterior muscle and is placed either between this two muscles or between the rib cage and the serratus anterior muscle.

13. The method according to claim 1, wherein at least one of the first electrode and the second electrode are moved into the thoracic region of the patient by a tunneling process comprising the following steps:
providing a tunneling tool located within a tube;
moving the tunneling tool and the tube concomitantly through the skin incision towards the intended implantation site of one of the first electrode and the second electrode until a distal end of the tube is located at the intended implantation site;
keeping the lube in place and removing the tunneling tool from the tube;
moving one of the first electrode and the second electrode through the tube towards its intended implantation site until it exits the tube at the intended implantation site;
keeping the one of the first electrode and the second electrode in place and removing the tube from the patient's body; and
removing the tube from the electrode.

14. The method according to claim 1, wherein at least one of the first electrode and the second electrode are moved into the thoracic region of the patient by a tunneling process comprising the following steps:
providing a tunneling tool and a tube, the tunneling tool having an electrode receiving portion in a distal region of the tunneling tool;
connecting one of the first electrode and the second electrode to the tunneling tool at the electrode receiving portion;
moving the tunneling tool, the one of the first electrode and the second electrode connected to the tunneling tool and the tube concomitantly through the skin incision towards the intended implantation site of the one of the first electrode and the second electrode until a distal end of the tube is located at the intended implantation site;
disconnecting the one of the first electrode and the second electrode from the tunneling tool;
keeping the one of the first electrode and the second electrode in place and removing the tunneling tool and the tube from the patient's body; and
removing the tube from the electrode.

15. A kit for implanting an implantable medical dev ice into a body of a human patient in need of such implantation, the kit comprising:
an implantable medical device configured to sense electrical signals from a heart of a patient and optionally to stimulate the patient's heart with electrical signals, the implantable medical device having at least two electrode connectors;
a first electrode connected to or connectable to a first of the electrode connectors of the implantable medical device and a second electrode connected to or connectable to a second of the electrode connectors of the implantable medical device; and
at least one tunneling tool configured to prepare an implantation path for at least one of the first electrode and the second electrode.

16. The kit of claim 15, wherein the kit further comprises an insertion catheter configured to receive the tunneling tool in its interior during an advancement of the tunneling tool through a body of a human patient for preparing an implantation path for at least one of the first electrode and the second electrode.

17. A method of treatment of a cardiac arrhythmia of a human patient with a cardiac pacing arrangement, the cardiac pacing arrangement comprising:
an implantable medical device configured to sense electrical signals from a heart of a patient and to stimulate the patient's heart with electrical signals, the implantable medical dev ice having at least two electrode connectors;
a first electrode connected to a first of the electrode connectors of the implantable medical device and a second electrode connected to a second of the electrode connectors of the implantable medical device;
wherein the human patient has a heart, a skin, a thorax and an abdomen, wherein the thorax is located in a thoracic region of the patient and the abdomen is located in an abdominal region of the patient,
wherein the cardiac pacing arrangement has been implanted into the human patient by a single skin incision into the patient's skin in an abdominal region of the patient.

18. The method according to claim 17. wherein the first electrode and the second electrode are implanted under the patient's skin:
i) on the thorax of the patient,
ii) between muscles of the patient in the thoracic region, or
iii) under muscles of the pattern in the thoracic region,
wherein a vector of an electric field between the first electrode and the second electrode crosses the biggest possible muscle mass of the patient's heart.

19. The method according to claim 17, wherein the implantable medical device carries out the following steps:
sensing electrical signals of the patient's heart with at least one of the first electrode, the second electrode and a housing of the implantable medical device;
evaluating the sensed electrical signals to detect a cardiac rhythm of lire patient's heart requiring therapy;
if a cardiac rhythm requiring therapy has been detected, delivering by at least one of the first electrode, the second electrode and the housing of the implantable medical device an electrical pulse once or in a cyclic manner, the electrical pulse being appropriate to therapeutically act upon the patient's heart.

20. The method according to claim 17, wherein a plurality of low-energy pulses are delivered if a bradyarrhythmia has been detected and a single high-energy pulse is delivered if a tachyarrhythmia has been detected.
